(19) United States
(12) Patent Application Publication
(10) Pub. No.: US 2022/0074786 A1
(43) Pub. Date: Mar. 10, 2022

(54) CONCENTRATE PROFILE AND LABELING

(71) Applicant: Radient Technologies Innovations Inc., Edmonton (CA)

(72) Inventors: Michael Cabigon, Edmonton (CA); Jim Seethram, Edmonton (CA); Steven Splinter, Edmonton (CA); Denis Taschuk, Edmonton (CA)

(21) Appl. No.: 17/239,774

(22) Filed: Apr. 26, 2021

Related U.S. Application Data
(63) Continuation of application No. PCT/IB19/58914, filed on Oct. 18, 2019.
(60) Provisional application No. 62/750,140, filed on Oct. 24, 2018.

(51) Int. Cl. G01G 19/415; A23L 33/105; B65C 9/40

(52) U.S. Cl. CPC G01G 19/415; A23L 33/105; G01N 33/02; A23V 2002/00; B65C 9/40

(57) ABSTRACT

Methods and systems for quantifying a variety of cannabis concentrate attributes relating to potency of a cannabis derivative product are disclosed. The methods and systems further generate a standard index rating to be affixed to a label for a cannabis concentrates and/or cannabis-infused products. The methods and systems include an extractor network, a product labeling system, and a user device coupled via a communication network. The product labeling system operable to analyze a cannabis product and receive additional input from a user application and store information relating to a cannabis product for consumers to review.

### CROSS-REFERENCE TO RELATED APPLICATION

The present patent application is a continuation of International Application No. PCT/IB2019/058914 filed Oct. 18, 2019, which claims the priority benefit of U.S. provisional patent application No. 62/750,140 filed Oct. 24, 2018, the disclosures of which are incorporated by reference herein.

### BACKGROUND OF THE INVENTION

### 1. Field of the Disclosure

The present disclosure is generally related to a method and system for the profiling and labeling of cannabis concentrate-infused product. More specifically, the present disclosure relates to a method and system for implementing a standardized rating system relating to edible cannabis products.

Moreover, the present disclosure provides a method and system for correlating user input to the profiling and labeling systems.

### 2. Description of Related Art

Cannabis is a genus belonging to the family cannabaceae. There are three common species of cannabis including Cannabis stavia, Cannabis indica, and Cannabis ruderalis. The genus cannabaceae is indigenous to Central Asia and the Indian subcontinent and has a long history of being used for medicinal, therapeutic, and recreational purposes. For example, cannabis is known to be capable of relieving nausea (such as that accompanying chemotherapy), pain, vomiting, spasticity in multiple sclerosis, and increase hunger in anorexia.

Cannabis contains a unique class of terpeno-phenolic compounds known as cannabinoids, or phytocannabinoids. The principle cannabinoids present in a cannabis can include Delta-9-tetrahydrocannabinolic acid (THCA) and cannabidiolic acid (CBDA). THCA does not include psychoactive properties on it's own, but when decarboxylated THCA becomes Delta-9-tetrahydrocannabinol (THC), which is a potent psychoactive cannabinoid. CBDA can be decarboxylated into cannabidiol (CBD), which is a major cannabinoid substituent in hemp cannabis. CBD is a non-psychoactive cannabinoid and is widely known to have therapeutic potential for a variety of medical conditions including, but not limited to, those described above.

Historical delivery methods for cannabis have involved smoking, or combusting, the dried cannabis plant material. Smoking can have adverse effects on the respiratory system via the production of potentially toxic substances. In addition, smoking is an inefficient mechanism which can delivers a variable mixture of active and inactive substances, many of which may be undesirable. Alternative delivery methods, such as ingesting, typically require extracts of the cannabis biomass, also known as cannabis concentrates or cannabis oils, to be created. Often, cannabis extracts are combined with a convenient pharmacologically acceptable or food-grade diluent, carrier, or excipient to produce a composition, which collectively may be known as cannabis derivative products or cannabis products. Such cannabis products can include, but are not limited to, cannabis topicals, edibles, and vaping products. Cannabis edibles can include cannabis-infused food or beverage products.

Ingestible products containing cannabis extract, as described above, have emerged as a popular and lucrative facet of the legalized cannabis market for both recreational and medicinal uses. However, the many formulations of cannabis extracts used in such products present a unique regulatory challenge for policy makers. Though ingestible, or consumable, cannabis products are often considered a safe, discreet, and effective means of attaining the desired therapeutic and/or intoxicating effects of cannabis without exposure to the potentially harmful risks of smoking, it is difficult for consumers to know the effect the products they consume will have. The most prominent difference between ingestion and inhalation of a cannabis extract is the delayed onset of the cannabinoid effect with ingestion. For example, consumers often do not understand this aspect of consumable cannabis product use and have been found to consume a greater than intended amount of cannabinoid before the cannabinoids have taken effect, often resulting in profoundly adverse effects.

The present disclosure relates to methods and systems for a profiling and labeling system for cannabis concentrates to be used in food and beverage products. The systems and methods described herein are able to provide consumers with a better understanding of the potency of an consumable cannabis product.

### SUMMARY OF THE CLAIMED INVENTION

Examples of the present disclosure provide systems and methods for analyzing a cannabis product for cannabinoid potency. In particular, a system for evaluating potency can include an extractor network, a product labeling system, and a user device communicably coupled with one another via a communication network. The product labeling system can receive cannabis concentrate data from the extractor network for use in an analysis of a cannabis-infused product. The cannabis potency can be standardized using an index rating and provided to consumers via a label on a cannabis product.

In addition to labeling the cannabis product, the systems and methods disclosed herein can provide consumers with increased amounts of information relating to the products they consume. This can allow consumers to better select the cannabis products they purchase and better anticipate the effects of the product once consumed.

### BRIEF DESCRIPTIONS OF THE DRAWINGS

FIG. 1 illustrates an exemplary network environment in which a system for cannabis concentrate profiling and labeling may be implemented.

FIG. 2 is a flowchart illustrating an exemplary method for labeling an edible cannabis product.

FIG. 3 is a flowchart illustrating an exemplary method for determining a potency index rating.

FIG. 4 is a flowchart illustrating an exemplary method for verifying the index rating determined in FIG. 3.

FIG. 5 is a flowchart illustrating an exemplary method for retrieving and uploading user rating data.

FIG. 6 is a flowchart illustrating an exemplary method for a user to request potency data, and upload resulting effects, of a cannabis product.

### DETAILED DESCRIPTION

The present disclosure presents a system and method for using a unified industry standard for the calculation of a strength index in the labeling cannabis concentrates and cannabis-infused edible products based on the potency, or strength (cannabinoid content), of the product. Furthermore, the present disclosure incorporates such potency rating system into a product labeling system for cannabis concentrate and cannabis-infused edible products.

The present disclosure illustrates a system and method for using an industry rating standard (such as, an International Cannabis Strength Units (ICSU)) for all cannabis concentrates and cannabis-infused products based on the potency (concentration of cannabinoids) of the product. A standardized rating system as disclosed herein can serve as a part of a consistent labeling system which informs the user the dosage and strength of the cannabis concentrate they intend to take. Additionally, the system described herein can enable prestige marketing through ingredient composition (for example: provide labeling for cannabis-infused edible products that are GMO free, organic, or fair trade).

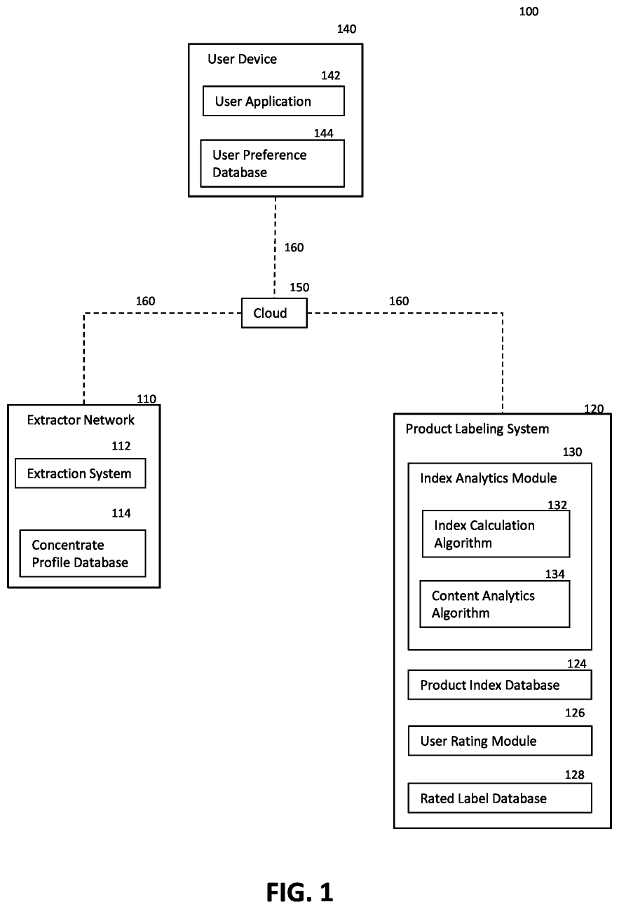

FIG. 1 illustrates an exemplary network environment in which a system for cannabis concentrate profiling and labeling may be implemented. This system 100 comprises at least an extractor network 110 which can have an extraction system 112 and a concentrate profile database 114 stored thereon. The extraction system 112 can be operable to extract a cannabis concentrate from a cannabis biomass. The concentrate profile database 114 can be operable to store information relating to each of various type of cannabis concentrates extracted. In at least one example, such information can be received from the extraction system 112 and stored on the concentrate profile database 114. In an alternative example, the information can be entered into the concentrate profile database 114 from a source other than the extraction system 112. Specifically, the concentrate profile database 114 can contain desired cannabis concentrate attributes including, but not limited to, a concentration or potency of cannabinoids (such as THC, CBD), a cannabinoid profile, a terpene profile, micro-testing including microbiological residues, chemical residues, pesticide residues, and heavy metal residues, for each type of cannabis concentrate produced.

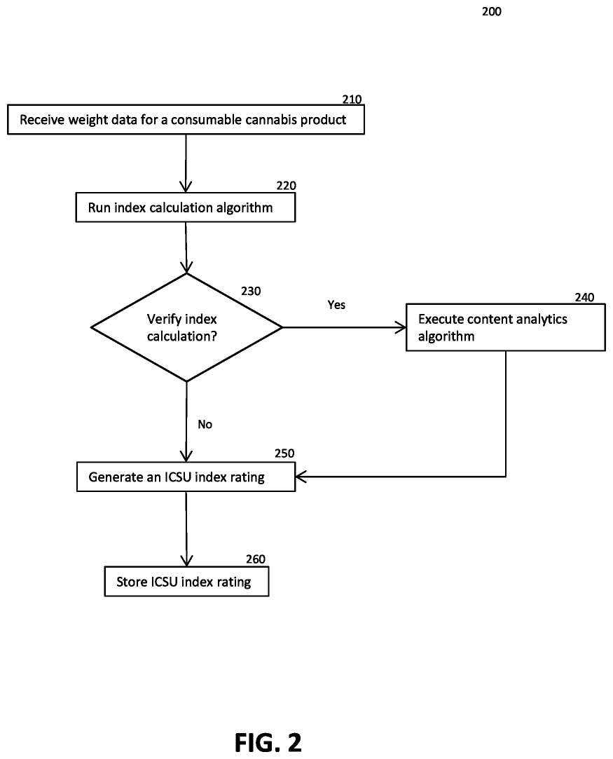

As described above, the system 100 can further comprise a product labeling system 120 having an index analytics module 130 operable to calculate a proprietary International Cannabis Strength Unit (ICSU) index number for each cannabis concentrate, or cannabis-infused product. The ICSU can be used to indicate strength of the cannabis concentrate or cannabinoids within the consumable cannabis product. The product labeling system 120 can also be operable to affix labels to the cannabis product with the index number and collect user ratings on the effects and user attributes related to product. The product labeling system 120 further comprises a software module operable to execute an index calculation algorithm 130, followed by a content analytics algorithm 134 to perform various correlations to ensure that the calculated ICSU index rating is consistent. The product labeling system 120 further comprises a device operable to measure the weight of each cannabis-infused product and sends the weight data to the index analytics module. The index analytics module 130 is operable to retrieve weight data of a cannabis concentrate or cannabis infused product from the weighing device and the cannabis concentrate data from the concentrate profile database 114 from the extractor network 110 for each type of cannabis concentrate infused in the cannabis product. The index calculation algorithm can use the concentration data along with the final weight of the consumable cannabis product to calculate the ICSU. The function of the index analytics module 130 is further described with respect to FIGS. 2 to 4.

The index analytics module 130 further comprises a content analytics algorithm 134 which can be executed following the index calculation algorithm 132 and uses analytical methods to ensure consistency in the index rating of the cannabis concentrate infused in the cannabis product. The index analytics module 130 module allows analytical methods including, but not limited to, liquid chromatography, gas chromatography or mass spectrometry, to determine the correlation between an ICSU index calculated by weight and the measured effective cannabinoid content. Once the ICSU rating is calculated, the product labeling system 120 can generate a product label having the ICSU index rating.

The product labeling system 120 further comprises a product index database 124 which contains information relating to various cannabis-infused products and the ICSU index number correlating to each product. Additionally, the product labeling system 120 can be communicatively coupled with a user device via a communication network 160. The product labeling system 120 further comprises a user rating module 126 that retrieves product rating data relating to the organoleptic properties (including, but not limited to, flavor), consistency, and effects for each consumable cannabis product having a label generated by the product labeling system 120. The product labeling system 120 further comprises a rated label database 128 which contains data relating to all labeled cannabis products and the product rating data for each type of product (that bears the label). The product information can be retrieved from a user application 142 accessible by the user device 140 and used to populate the rated label database 128 of the product labeling system 120 or send the product rating data for the product to a user.

The system 100 further comprises a user device 140 communicably coupled with the product labeling system via a communication network 160. The user device 140 can enable users to determine, or look up, the flavor and basic attributes of the consumable cannabis-infused product, or to look up for reviews and ratings of a product based on the product label. As stated above, the system 100 further comprises a user application 142, or app, on the user device 140. The user application 142 can be operable to prompt the user to rate a labeled product and send the rating to the user rating module 126 of the product labeling system 120. In addition, the user application 142 can be operable to receive the ICSU index rating and user product ratings from the user rating module 126 and displays it to the user via a display of the user device 140. In at least one example, the user application 142 can be used to access a user account which can allow users to keep a log of cannabis products they have used. The user device 140 can include a user preference database 144 operable to store registered user IDs relating to the user account and the user's preferred products and range of ICSU.

The functioning of the index analytics module is explained with reference to FIG. 2. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

FIG. 2 illustrates an exemplary method 200 for labeling a consumable cannabis product. The method 200 can be completed using the index analytics module 130, described above. The method 200 can begin at block 210 where the index analytics module 130 receives weight data from a weighing scale, the weight data relating to each consumable cannabis concentrate-infused product. The weight of the cannabis product is then used to calculate the unit product weight for each discrete cannabis product. At block 220, the index analytics module 130 can execute an index calculation algorithm. In at least one example, the index calculation algorithm can use the weight data obtained at block 210 as well as cannabis concentrate data provided by an extractor network 110, as described above, to perform the analysis.

At block 230 the method 200 can elect whether to verify the index calculation provided by the index calculation algorithm. If the method 200 determines to verify the calculation, the method can proceed to step 240 and run a content analytics algorithm. The content analytics algorithm can be used to perform various correlations to check and is operable to ensure consistency in cannabis concentrate index calculations. Once the index is verified, the method 200 can proceed to block 250, where an ICSU index is generated. The ICSU index generated at block 250 will be subsequently transferred onto a label and affixed to the product. At block 260, the index analytics module can populate the product index database, described above, with the ICSU index generated.

In the alternative, the method 200 elects not to verify the index calculation, the method can proceed directly from block 230 to block 140 and generate an ICSU based on the calculations performed by the index calculation algorithm.

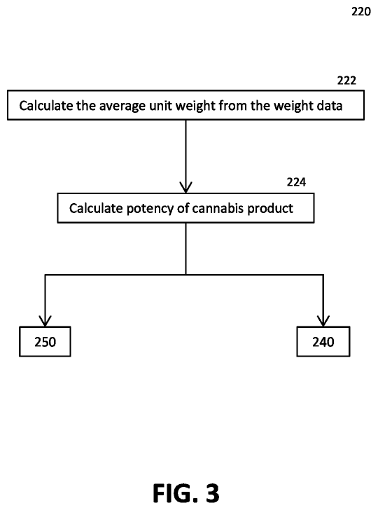

A detailed description of the index calculation algorithm is explained with reference to FIG. 3, which is a flowchart illustrating an exemplary method 300 for determining a potency index rating. Specifically, method 300 may be performed as part of block 220 of FIG. 2. The index calculation algorithm can begin at block 222 where the average unit weight is calculated based on the weight data and concentrate profile data received from the index analytics module. At block 224, the index calculation algorithm calculates, using a weight percentage formula, the percentage of cannabinoid potency content based on the overall unit weight of the cannabis product. The resulting potency can then return to method 200 and proceeds either to block 240 where the content analytics algorithm is executed, or to block 250 where an ICSU index rating for the product is generated.

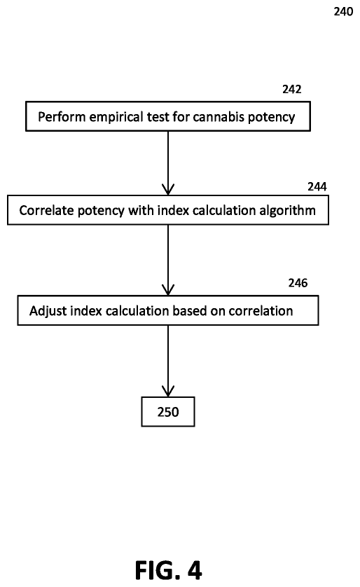

A detailed description of the functioning of the content analytics algorithm is explained with reference to FIG. 4, which is a flowchart illustrating an exemplary method for verifying the index rating determined in FIG. 3. The content analytics algorithm can begin at block 242 where the product labeling system, described above, empirically analyzes the cannabis product to determine, at least one of a concentration, or potency, of cannabinoids (including, but not limited to, THC, and CBD) of the product, a cannabinoid profile, a terpene profile, micro-testing including microbiological residues, chemical residues, pesticide residues, heavy metal residues, and combinations thereof. At block 244, the content analytics algorithm can then proceed to run a correlation between the outcome of the index calculation algorithm, as described in FIG. 3, and the empirical results. At block 246, the content analytics algorithm can determine an adjusted index calculation for the cannabis concentrate product based on the correlation. The content analytics algorithm can then proceed to block 250, where an ICSU is generated based on the adjusted index calculation. Using an adjusted index calculation can provide additional accuracy with respect to the ICSU index rating of the product, due to the use of empirical test results.

After an ICSU index rating has been calculated for the consumable cannabis product, the information can be stored in a product index database as described with respect to FIG. 1. An exemplary product index database entry is provided as Table 1, below. It should be understood that the exemplary entry provided as Table 1 is provided merely for the purposes of this description and is not to be considered as limiting.

TABLE 1

| Product ID | Potency Content | ICSU Index |
|---|---|---|
| 0001 | THCA | 11.7 |
| 0002 | THCA, CBGA | 15.4 |
| 0003 | CBCA | 8.9 |

As shown in Table 1, the cannabis product can be provided a product identification number, or product ID. The product ID can be used to store information relating to a specific cannabis product. In at least one example, the product ID can be used to identify information such as the grower of the cannabis biomass used to create the product, the extractor who extracted the cannabinoid concentrate, and manufacturer of the final cannabis product. Additional information can include, but is not limited to, location where the cannabis crop was grown and what nutrients or growth products were used during the maturation of the plants. The product index database can also include a potency content related to each product ID, indicating which cannabinoids are present in the cannabis product. Finally, the product index database can provide the ICSU index number relating to each product ID. In at least one example, the product index database can be accessible by users via a user application, as described above.

In addition to being stored in the product index database, the ICSU rating for the cannabis product is can be printed on a label to be affixed to the product, such that a user can view the ICSU rating prior to consuming the cannabis product. Such ICSU rating allows users a better sense of what cannabinoids are in the product they are purchasing or consuming, and what effects they can anticipate. In at least one example, as described above, a user can access information relating to labeled cannabis products via a user application on a user device.

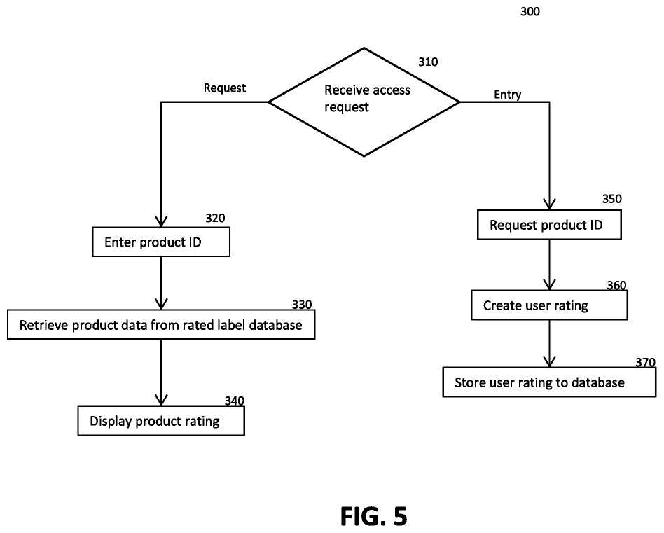

Furthermore, after consuming the product, users can provide a rating and list the effects the cannabis product had on them for upload to a product rated label database as shown in FIG. 1. Specifically, a method 300 for using a user rating module to send and receive user rating data is explained with reference to FIG. 5, which is a flowchart illustrating an exemplary method for retrieving and uploading user rating data. The method 300 can begin at block 310 where a user rating module receives an access request from a user application on a device. The device can be any personal computing device operable to communicate with the user rating module of the product labeling system described above. The request can either be a request for product information or a request to create an entry relating to a cannabis product. At block 310, the user rating module determines if the user has requested product rating data or created user rating entry.

At block 320, if the request is to retrieve a product rating data, the user rating module requests the product ID. In at least one example, a product ID number, or code, can be located on a label of the consumable cannabis product. In an alternative embodiment, the user rating module can accept a photograph of the cannabis product packing including, but not limited to, a photo of the label, a photo of a barcode, or any other image. At block 330, the user rating module can look up the product ID in the rated label database, described above. At block 340, if a product label is found in the database, the user rating database can display the average rating. The average rating can include, but is not limited to, the ICSU rating index, other user ratings, a list of effects the consumable corresponding to that product ID had on other users, and combinations thereof.

In the alternative, if the user rating module determines that the user has requested to create an entry relating to a cannabis product, the method 300 can proceed to block 350. At block 350, the user rating module can a product ID for the cannabis product. As described above, the product ID can be provided either by entering a number, or code, or uploading an image of the product label. At block 360, the user rating module can create and populate an entry for the rated label database with the product rating data. At block 370, the user rating module can store the product rating data as a new entry in the rated label database.

An exemplary entry from the rated label database is provided in Table 2, below. It should be understood that the exemplary entry provided as Table 2 is provided merely for the purposes of this description and is not to be considered as limiting.

TABLE 2

| Product Label ID | Average User Rating |
|---|---|
| USVA 345789 | 80.2 |
| CABC 233490 | 84.5 |
| CAOT 293489 | 45.2 |

As shown, the rated label database can include the product identification number, or product ID. As described above, the product ID provide a user with a large amount of information regarding the origin and processing of the final cannabis product. The database can also include the user product rating data corresponding to each product ID. In at least one example, the user application can include a description of the user rating system and provides an explanation of how the user rating correlates to the potency, effects, and taste of the consumable cannabis products.

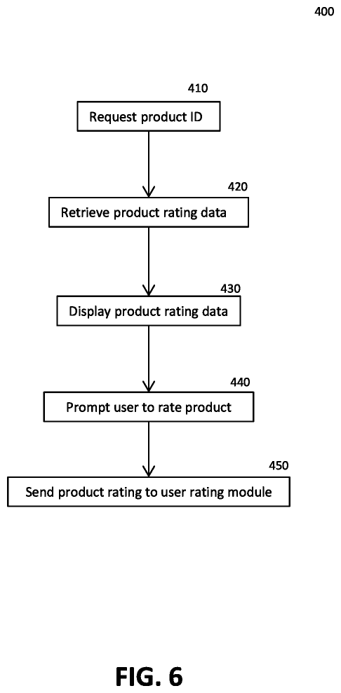

A user application, or user app, can allow a user to retrieve information relating consumable cannabis products and provide ratings to products they have used. A method 400 for retrieving and uploading information relating to a cannabis product using a user app is explained with reference to FIG. 6, which is a flowchart illustrating an exemplary method for a user to request potency data, and upload resulting effects, of a cannabis product. The method 400 can be executed on a user app stored on a user device. Each user can have an account with a unique login such that preferences can be stored based on the user identification. The method 300 can begin at block 410 where the user app asks the user to enter the product ID to be searched. As described above with respect to FIG. 5, the product ID can be entered into the user app using a variety of methods. At block 420, the user app can send the product ID to the user rating module, as described above the user rating module can look up the average product rating data and ICSU index rating for the item corresponding the product ID provided. At block 430 the user app can display the average rating data stored in the rated label database and the ICSU index rating. In at least one example, a user can access the rating from the user app while purchasing products. As such, the rating system can assist a user in making consumable cannabis product purchase decisions. Specifically, the ICSU index rating can provide users with the cannabinoid content (for example THC or CBD) of the cannabis product allowing users to better understand the effects of the product.

At block 440, the user app can prompt the user to rate the labeled product which they have just searched. If the user has consumed the consumable cannabis product, they can create an entry including a rating and a description of the effects of the product, and the longevity of the effects. At block 450, the user app can send the product rating data to the user rating module for storage as described with respect to FIG. 5.

As described above, the user app can include an account which has a unique login for each user. The user app can then store information relating to the consumable cannabis product each user has used to a user preferences database. The user preferences database can store data including, but not limited to, user identification, including at least a user age, user purchase history, user rating history, and the like. An exemplary entry from a user preferences database is provided as Table 3. It should be understood that the exemplary entry provided as Table 3 is provided merely for the purposes of this description and is not to be considered as limiting.

TABLE 3

| User ID | Adam Apple | Product History | ICSU |
|---|---|---|---|
| | | 0013 | 11.47 |
| | | 0037 | 20.38 |

Table 3 illustrates an entry for a user, Adam Apple. The database can include a purchase, or product history, for the user. For example, Table 3 indicates that user Adam Apple has used product ID 0013 and 0037 in the past. The exemplary entry also includes the ICSU index rating for the products that user Adam Apple has used. While the example provided above does not include information relating to user ratings, it should be understood that the user preference history can include a user rating entry for each product ID which the user has consumed in the past.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claims.

What is claimed is:

1. A method of potency labeling for cannabis products, the method comprising:
receiving cannabis concentrate data regarding a cannabis product, the cannabis concentrate data sent to a product labeling system;
receiving a weight of the cannabis product at an index analytics module;
executing an index calculation algorithm of an index analytics module to obtain an index rating, wherein the index rating is based on the cannabis concentrate data and the weight of the cannabis product; and
generating a label reflecting the index rating, wherein the generated label is configured to affix to the cannabis product.

2. The method of claim 1, wherein executing the index calculation algorithm includes calculating an average unit weight based on the weight of the cannabis product and the cannabis concentrate data.

3. The method of claim 2, wherein executing the index calculation algorithm includes calculating a cannabinoid potency based on the average weight unit, wherein the generated label further reflects the calculated cannabinoid potency.

4. The method of claim 3, further comprising correlating the cannabinoid potency to results of an empirical test.

5. The method of claim 1, further comprising verifying the index rating by:
analyzing the cannabis product to determine a cannabinoid concentration;
comparing the cannabinoid content to the index rating; and
adjusting the index rating based on the comparison to obtain a verified index rating.

6. The method of claim 1, wherein the cannabis concentrate data includes a profile associated with the cannabis product.

7. The method of claim 6, wherein the profile includes at least one of a cannabinoid profile, a terpene profile, micro-testing including microbiological residues, chemical residues, pesticide residues, and heavy metal residues.

8. The method of claim 1, further comprising selecting the index rating using a standardized rating system.

9. The method of claim 1, further comprising receiving a user rating for the cannabis product over a communication network from a user device, and storing the user rating in a database in association with the cannabis product, wherein the generated label further reflects the user rating.

10. A system of potency labeling for cannabis products, the system comprising:
an interface that
receives cannabis concentrate data regarding a cannabis product; and
receives a weight of the cannabis product at an index analytics module;
an index analytics module that includes an index calculation algorithm executable by a processor to obtain an index rating, wherein the index rating is based on the cannabis concentrate data and the weight of the cannabis product; and
a printer that prints a label reflecting the index rating, wherein the label is configured to affix to the cannabis product.

11. The system of claim 10, wherein the index calculation algorithm is further executable to calculate an average unit weight based on the weight of the cannabis product and the cannabis concentrate data.

12. The system of claim 11, wherein the index calculation algorithm is further executable to calculate a cannabinoid potency based on the average weight unit, wherein the label further reflects the calculated cannabinoid potency.

13. The system of claim 12, wherein the index calculation algorithm is further executable to correlate the cannabinoid potency to results of an empirical test.

14. The system of claim 10, further comprising a content analytics algorithm executable to verify the index rating by:
analyzing the cannabis product to determine a cannabinoid concentration;
comparing the cannabinoid content to the index rating; and
adjusting the index rating based on the comparison to obtain a verified index rating.

15. The system of claim 10, wherein the cannabis concentrate data includes a profile associated with the cannabis product.

16. The system of claim 15, wherein the profile includes at least one of a cannabinoid profile, a terpene profile, micro-testing including microbiological residues, chemical residues, pesticide residues, and heavy metal residues.

17. The system of claim 10, wherein the index calculation algorithm is further executable to select the index rating using a standardized rating system.

18. The system of claim 10, further comprising a user rating module that receives a user rating for the cannabis product over a communication network from a user device; and a label database that stores the user rating in association with the cannabis product, wherein the generated label further reflects the user rating.

19. A non-transitory, computer-readable storage medium, having embodied thereon a program executable by a processor to perform a method of potency labeling for cannabis products, the method comprising:
receiving cannabis concentrate data regarding a cannabis product, the cannabis concentrate data sent to a product labeling system;
receiving a weight of the cannabis product at an index analytics module;
executing an index calculation algorithm of an index analytics module to obtain an index rating, wherein the index rating is based on the cannabis concentrate data and the weight of the cannabis product; and
generating a label reflecting the index rating, wherein the generated label is configured to affix to the cannabis product.
